(19) United States
(12) Patent Application Publication
(10) Pub. No.: US 2021/0050081 A1
(43) Pub. Date: Feb. 18, 2021

(54) SYSTEMS AND METHODS FOR ON-PREMISES PRESCRIPTION MEDICATION SELECTION, ORDERING, AND PAYMENT

(71) Applicants: Spencer Malkin, Boca Raton, FL (US); Mate Benko, Fort Lauderdale, FL (US)

(72) Inventors: Spencer Malkin, Boca Raton, FL (US); Mate Benko, Fort Lauderdale, FL (US)

(21) Appl. No.: 16/540,775

(22) Filed: Aug. 14, 2019

(51) Int. Cl. G16H 20/10; G06Q 30/06

(52) U.S. Cl. CPC G16H 20/10; G06Q 30/0641; G06Q 30/0639; G06Q 30/0611

(57) ABSTRACT

Systems and methods for prescription medication selection, ordering, and payment are disclosed. Medication information may be received via a first computing device located at a healthcare provider premises. The medication information for a patient's medication may then be sent to a plurality of medication providers. One or more offers to fulfill the medication may be received from the plurality of medication providers. The one or more offers may then be sent to a second computing device located at the healthcare provider premises. A selection of an offer may be received from the second computing device. The selection of the offer may be indicated to the medication provider associated with the selected offer.

### TECHNICAL FIELD

This disclosure generally relates to prescription medications and, more particularly, to on-premises prescription medication selection, ordering, and payment.

### BACKGROUND

Receiving a prescription for medication by a healthcare provider is a quite common occurrence. This may include one-off prescriptions to address the occasional illness or more regular prescriptions relating to a chronic condition. Yet the process of filling a prescription can include numerous frustrations and inefficiencies. For example, this may include several trips to a local pharmacy to submit the prescription and to later pick up and pay for the prescribed medication. As another example, the pricing for medications often lacks transparency to the consumer, with different pharmacies offering different prices for the same medication. Not only may retail prices differ, but the prices negotiated between various pharmacies and pharmacy benefit managers may also differ. Coupons and rebates for certain drugs also complicate consumer purchasing decisions. The process is yet further complicated when mail-order pharmacies are considered in addition to brick-and-mortar pharmacies.

These and other shortcomings are addressed in the present disclosure.

### SUMMARY

Disclosed herein are system and methods for on-premises prescription medication selection, ordering, and payment. In an example method, medication information is received via a first computing device located at a healthcare provider premises. The medication information may indicate a medication associated with a patient. The medication information is sent to a plurality of medication provider systems associated with respective medication providers. One or more offers are received from respective medication provider systems of the plurality of medication provider systems. An offer may comprise an offer by the associated medication provider to fulfill the medication. The one or more offers are sent to a second computing device located at the healthcare provider premises. A selection of an offer of the one or more offers is received from the second computing device. The selected offer may be associated with a selected medication provider system. An indication that the patient has selected the offer associated with the selected medication provider system is sent to the selected medication provider system.

Implementations of any of the described techniques may include a method or process, an apparatus, a device, a machine, a system, or instructions stored on a non-transitory computer-readable storage device. The details of particular implementations are set forth in the accompanying drawings and description below. Other features will be apparent from the following description, including the drawings, and the claims.

### BRIEF DESCRIPTION OF DRAWINGS

The accompanying drawings, which are incorporated in and constitute a part of this specification, illustrate embodiments and together with the description, serve to explain the principles of the methods and systems:

FIG. 1 illustrates an example system and network configuration according to an embodiment of the present disclosure;

FIG. 2 illustrates an example block diagram according to an embodiment of the present disclosure;

FIG. 3 illustrates an example block process diagram according to an embodiment of the present disclosure;

FIG. 4 illustrates an example schematic diagram according to an embodiment of the present disclosure; and

FIG. 5 illustrates an example method flow chart according to an embodiment of the present disclosure.

Aspects of the disclosure will now be described in detail with reference to the drawings, wherein like reference numbers refer to like elements throughout, unless specified otherwise.

### DETAILED DESCRIPTION

The systems and methods of the present disclosure relate to on-premises prescription medication selection, ordering, and payment. A healthcare provider may enter medication information into a healthcare provider system located at the healthcare provider's premises. The medication information may indicate a medication that the healthcare provider considers prescribing or is prescribing for a patient. The healthcare provider system may send the medication information to a prescription processing system. There, the prescription processing system may pass along the medication information to a plurality of medication providers, such as mail-order pharmacies. Some or all of the medication providers may return with respective offers to fulfill the prescription for the medication for the patient. For example, an offer may include a price for the medication from the particular medication provider.

The offers may be received by a point of sale device also located at the healthcare provider premises. For example, the medication information may be entered and sent to the medication providers and the offers may be received at the point of sale device during the patient's same healthcare visit (e.g., doctor's office visit). The patient may select an offer using the point of sale device. The selection may be returned to the medication provider that made the selected offer. The patient may submit payment to the medication provider for the medication using the point of sale device at generally the same time as selecting an offer. The patient may additionally or alternatively submit payment to the healthcare provider for the healthcare visit using the point of sale device, also at generally the same time. After being notified that the patient has selected their offer, the associated medication provider may fulfill the order for the medication.

A number of technical benefits may be realized by the present disclosure. Oftentimes, it is difficult for a patient to assess the cost in general for a medication without extensive effort. It is even more challenging for a patient to assess the current costs for the medication right when the medication is contemplated or prescribed by the patient's healthcare provider. The numerous medication providers that may potentially fulfill the prescription, as well as their associated prices and other terms, represent simply more variables and data than a person could practically research and track, even when the patient has access to a computer.

In the present disclosure, the prescription processing system implements communications between the patient, the patient's healthcare provider, and medication providers such that the patient may be almost immediately presented with medication prices from numerous respective medication providers willing to fulfill the prescription, all while still at the healthcare provider's premises. Using the same device at the healthcare provider's premises (and even using the same website or application), the patient may select the most attractive price and submit payment for the medication. As will be understood from the above, this offers a “one-stop-shop” for the patient. Indeed, by the time the patient leaves the healthcare provider's premises, the patient will have already priced the medication, placed an order for a selected medication provider to supply the medication, and paid for the medication.

The technical process is further streamlined in that the healthcare provider may submit the medication to the prescription processing system using the same website or application as that used by the patient to perform the above. Yet further streamlining the technical process, the prescription processing system may provide an interface (e.g., a website portal) that medication providers may engage to receive price requests from patients, return prices in response to price requests, and receive medication orders and payments from patients. By providing a platform (e.g., website) common to the healthcare provider, the patient, and the medication providers, the prescription processing system improves electronic communication and transactions between the parties.

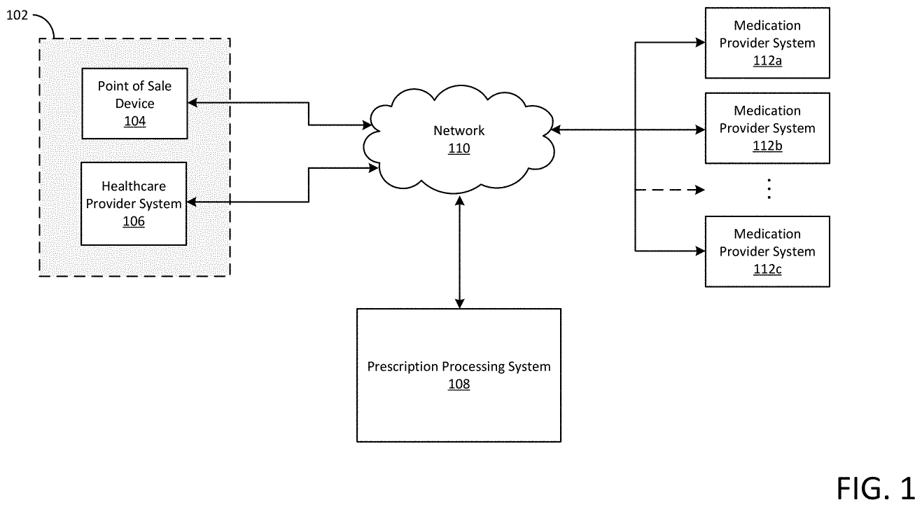

FIG. 1 illustrates an example hardware and network configuration in which the systems and methods described herein may be implemented. A healthcare provider system 106 and a point of sale device 104 may be located at a healthcare provider premises 102, such as a doctor's office. A healthcare provider may enter a medication (e.g., a prescription) for a patient into the healthcare provider system 106, which is sent to a prescription processing system 108. The prescription processing system 108 may indicate the medication to one or more medication provider systems 112a-c (which will be referred to generically as medication provider system(s) 112). The medication provider systems 112 may return respective offers for fulfilling a prescription for the medication back to the prescription processing system 108. The offers may be sent to the point of sale device 104 for the patient to select one of the offers. The prescription processing system 108 may transmit the selection to the particular medication provider system 112 associated with the selected offer. Payment to the selected medication provider system 112 for the medication may be entered by the patient via the point of sale device 104 at the healthcare provider premises 102. Communication between the various components may be via a network 110.

The healthcare provider premises 102 may be associated with a healthcare provider. A healthcare provider may include a physician, but may also include a nurse, a physician assistance, a nurse practitioner, or other type of healthcare practitioner. A healthcare provider may include a dentist. Generally, a healthcare provider may be one with prescription authority in the relevant jurisdiction. In some aspects, a healthcare provider may include a veterinarian, particularly those authorized to write prescriptions for animals. The healthcare provider premises 102 may comprise a medical facility or space in which patients are seen by a healthcare provider. This may include a physician's office, a clinic, or a hospital. The healthcare provider premises may encompass one or more examination rooms, a healthcare provider's office space, a reception area, and/or a waiting room.

The healthcare provider system 106 may comprise one or more computing devices configured to receive medication information (e.g., a prescription) entered by a healthcare provider. The healthcare provider system 106 may include a personal computer or a handheld computing device, such as a tablet computer. The healthcare provider system 106 may also include one or more off-premises computing devices, such as one or more servers, in communication with one or more on-premises computing devices. For example, a healthcare provider may directly interact with a personal computer at the healthcare provider premises 102 to enter a prescription and this prescription may be uploaded to an off-premises server of the healthcare provider system 106. The on-premises personal computer and the external servers may collectively form the healthcare provider system 106.

The point of sale device 104 may be located at the healthcare provider premises 102 and configured for interaction with a patient or associated individual. The point of sale device 104 may comprise a computing device. For example, the point of sale device 104 may comprise a personal computer, a handheld computer (e.g., a tablet computer), a computer console, or a computer kiosk. In some aspects, the point of sale device 104 may comprise a general use computing device. In other aspects, the point of sale device 104 may comprise a dedicated computing device. That is, the point of sale device 104 may be substantially dedicated to selection, ordering, and payment for prescription medication. For example, the point of sale device 104 may be restricted, from a patient's point of view, to run only application(s) or software relating to selection, ordering, and payment for prescription medication.

The point of sale device 104 may present one or more offers from one or more medication providers to fulfill the patient's prescription. The point of sale device 104 may receive an input from a patient indicating a selection of one such offer. The point of sale device 104 may facilitate payment by a patient for fulfillment of the selected offer and/or for services rendered by the healthcare provider. For example, the point of sale device 104 may be configured with a card (e.g., credit card) reader. Said card reader may be detachable, such as a portable credit card reader connected to (e.g., wirelessly or via wired/hardware connection) a tablet computer. As another example, the point of sale device 104 may be configured to receive a text input for payment, such as credit card number or bank account information.

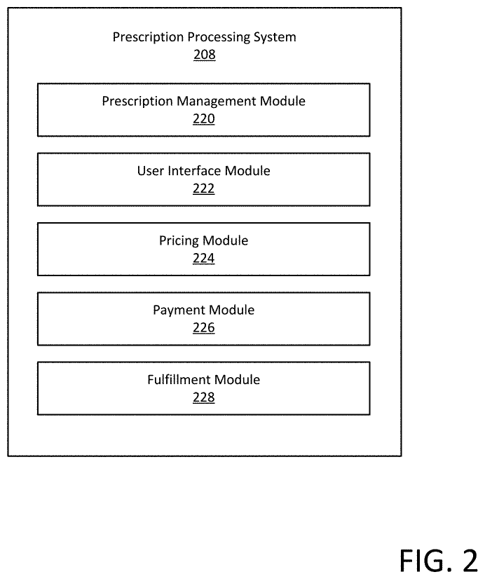

The prescription processing system 108 may implement a number of the functions and techniques described herein. The prescription processing system 108 may generally coordinate interactions between the healthcare provider, the patient, and the medication providers. The prescription processing system 108 may comprise one or more computing devices. For example, the prescription processing system 108 may comprise one or more networked servers. The prescription processing system 108 may comprise one or more networked storage devices or systems, such as one or more database servers. The prescription processing system 108 shall be discussed in further detail in relation to FIG. 2.

The medication provider systems 112 may be associated with respective medication providers. A medication provider may include a pharmacy, a pharmacy chain or brand, a brick-and-mortar pharmacy, a mail-order pharmacy, an online pharmacy, or a tele-pharmacy. Some medication providers may exhibit several of these characteristics, such as a pharmacy company that operates a number of brick-and-mortar pharmacies and also provides mail-order pharmacy services. A medication provider system 112 may receive medication information and respond to the medication information with an offer to provide the medication at a specified price. A medication provider system 112 may receive an order and associated payment for a medication and fulfill such an order. A medication provider may comprise or be associated with a medication distribution system. A medication distribution system may package and deliver the medication to the patient, such as via mail to the patient's residence.

A medication provider system 112 may comprise one or more computing devices, such as one or more networked servers. In some aspects, a medication provider system 112 may comprise a computer system distinct from the prescription processing system 108. For example, the prescription processing system 108 may be implemented using a first set of one or more networked computer systems and a medication provider system 112 may be implemented using a different, second set of one or more networked computer systems. In other aspects, the prescription processing system 108 and the medication provider systems 112 may be integrated, at least in part. For example, the prescription processing system 108 may comprise logical components associated with respective medication provider systems 112. In this case, communications between a medication provider system 112 and other components (e.g., modules) of the prescription processing system 108 may be internal to the prescription processing system 108.

The network 110 may comprise one or more public networks (e.g., the Internet) and/or one or more private networks. A private network may include a wireless local area network (WLAN), a local area network (LAN), a wide area network (WAN), a cellular network, or an intranet. The network 110 may comprise wired network(s) and/or wireless network(s).

FIG. 2 illustrates a block diagram of a prescription processing system 208. The prescription processing system 208 may be the same as or similar to the prescription processing system 108 in FIG. 1 in at least some aspects. The prescription processing system 208 comprises a plurality of modules, including a prescription management module 220, a user interface module 222, a pricing module 224, a payment module 226, and a fulfillment module 228. Each shall be described in turn.

The prescription management module 220 may perform medication information and/or prescription intake to the prescription processing system 208. The prescription management module 220 may receive medication information (e.g., a prescription) from a healthcare provider system (e.g., the healthcare provider system 106 of FIG. 1). The medication information may indicate the medication and dosage, the prescribing healthcare provider, and the patient. The medication information may include, or be sent along with, medical record information. The medication information may be in the form of an electronic prescription. The medical record information may be likewise in the form of an electronic medical record. Incoming medication information may be in the form of an incoming fax or incoming phone call, such as a fax or phone call from a healthcare provider. The medication information may indicate any benefits (e.g., prescription benefits) of the patient. The prescription management module 220 may manage recurring prescriptions and/or refills. For example, a request for offers for a prescribed medication may be sent to medication providers when a refill is available.

In some aspects, the prescription management module 220 may maintain information relating to alternative or otherwise related medications for a given medication. For example, the prescription management module 220 may receive medication information (e.g., a prescription) for a particular medication and the prescription management module 220 may determine one or more alternative medications that the healthcare provider may prescribe to the patient rather than the initial medication indicated in the prescription. The alternative medication(s) may include therapeutically similar medications. Additionally or alternatively, the prescription management module 220 may determine one or more alternatives with respect to dosage form or formulation. The alternative medication(s), etc. may be presented back to the healthcare provider and the healthcare provider may select one or more (or none) of the alternatives. The healthcare provider may indicate whether he or she wishes to receive back such suggested alternatives when submitting medication information to the prescription processing system 208.

The medication information may be provided to the prescription management module 220 in database form or as part of a database. The prescription management module 220 may process the database, which may include converting the database to another data format and/or extracting the medication information. The prescription management module 220 may coordinate or initiate one or more of the other modules of the prescription processing system 208. For example, after receiving and processing medication information, the prescription management module 220 may initiate the pricing module 224.

The pricing module 224 may implement functions relating to requesting for and receiving medication pricing offers from one or more medication providers. The pricing module 224 may identify one or more medication providers that may potentially fill a prescription for a specified medication. This may be based on a database of medication providers and their respective characteristics. A characteristic may include the medications available for supply by a medication provider. For example, some medication providers may supply certain specialty medications while others may not. A medication provider characteristic may include the regions that a medication provider serves or ships to. A medication provider characteristic may include the drug benefits plans or pharmacy benefit managers associated with the medication provider. For example, some medication providers may honor certain benefits plans or be preferred/in-network providers for certain benefits plans.

The pricing module 224 may track any benefits information for the patient, including benefits information that is included with received medication information. The pricing module 224 may also track any discounts, coupons, and rebates associated with the medication (and in general for numerous other medications). The discounts, coupons, and rebates may be cross-listed with the medication providers, as some of the discounts, coupons, or rebates may be applicable to some medication providers but not others.

The pricing module 224 may transmit medication information to the medication provider systems (e.g., the medication provider systems 112 of FIG. 1) associated with the respective identified medication providers. Transmitting the medication information may be regarded as a request for offers to supply the indicated medication. The medication information may be the same as or similar to the medication information received from the healthcare provider system in at least some aspects. The medication information sent to the medication provider systems may exclude, at least in part, personal information (e.g., patient information and medical record information) or include, at least in part, such information. The medication information sent to the medication provider systems may include benefits information for the patient. An offer sent back from a medication provider may be based on the benefits information, such as the price of the medication under the patient's benefits plan.

Based on the medication information sent to the medication provider systems, the pricing module 224 may receive “offers to fill” from respective medication provider systems. An offer may include the price at which the medication provider will supply the medication to the patient. An offer may include other terms, such as shipping costs and shipping times. More than one offer may be received from a single medication provider system, such as offers under various combinations of one or more benefits plans, rebates, discounts, and coupons. Multiple offers from a single medication provider system may also be with respect to the period of time covered by an order of the medication, such as whether a patient is supplied for a month's worth of dosages or three months' worth of dosages. Multiple offers from a single medication provider system may also be with respect to dosage form or medication formulation.

The pricing module 224 may transmit the offers received from the medication provider systems to a point of sale device (e.g., the point of sale device 104 of FIG. 1) for the patient to view and consider. The point of sale device may be located at the healthcare provider's premises. The patient may use the point of sale device to select an offer. The pricing module 224 may receive the offer selection from the point of sale device. The offer selection may be in the form of a prescription from the healthcare provider or include such a prescription.

The fulfillment module 228 may generally implement functions to cause the medication provider associated with the selected offer to deliver the medication to the patient, i.e., fulfill the prescription. The fulfillment module 228 may process the offer selection received by the pricing module 224. The fulfillment module 228 may generate and transmit a medication order to the medication provider system associated with the selected offer. The medication order may comprise the prescription for the medication. The medication provider may receive the order and communicate such order to an associated distribution center, which may package and deliver the medication to the patient.

The payment module 226 may generally implement function relating to payments at various stages in the steps described herein for medication selection, ordering, and payment. Accordingly, the payment module 226 may operate in conjunction with various other modules of the prescription processing system 208.

The payment module 226 may implement payment functions associated with the offer selection received by the pricing module 224 from the point of sale device. The payment module 226 may receive payment information for the selected offer that was entered via the point of sale device. “Payment information” may be variously referred to herein as simply “payment”. Payment information received from the point of sale device may comprise a credit card number, a bank account number, or other type of account identifier.

The payment module 226 may implement payment functions relating to payment to the medication provider associated with the selected offer, i.e., the medication provider that is to deliver the medication to the patient. The payment to the medication provider may be transmitted along with the medication order initiated by the fulfillment module 228 to the medication provider. The payment transmitted to the medication provider may include forwarding the payment information received from the patient via the point of sale device. In other instances, the prescription processing system 208 may process the payment information (e.g., credit card number) from the patient and make a separate payment (e.g., fund transfer) to the medication provider.

The payment module 226 may implement payment functions relating to payment to the healthcare provider for services rendered to the patient. For example, the patient may be required to provide a co-payment to the healthcare provider for the office visit. The payment to the healthcare provider may be made via the point of sale device. The payment may be made generally at the same time as selecting an offer and submitting payment to the medication provider, such as during the same use session of the point of sale by the patient. Payment to the healthcare provider and payment to the medication provider may be performed as separate transactions. The payment module 226 may transmit or otherwise effectuate the payment to the healthcare provider.

The user interface module 222 may implement functions generally relating to information input to the prescription processing system 208 by the healthcare provider and patient and information output to the healthcare provider and patient by the prescription processing system 208. For example, the user interface module 222 may implement a user interface at a healthcare provider system. The healthcare provider may enter medication information via the user interface. As another example, the user interface module 222 may implement a user interface at a point of sale device, such as for a patient to select an offer and enter payment information.

The user interface module 222 may comprise one or more web servers configured to host one or more websites that may be accessed at the healthcare provider system or point of sale device. The healthcare providers and patients may interact with the prescription processing system 208 via a common website. In aspects in which the healthcare provider system and/or the point of sale device run local application(s) to effectuate their various functions, the user interface module 222 may interact with said applications to transmit and receive data between the prescription processing system 208 and the healthcare provider system or point of sale device.

In some aspects, the user interface module 222 may implement a user interface associated with the medication providers. Medication providers may receive medication information via the user interface, such as medication information indicating that a patient requests offers from the medication providers to fulfill the patient's prescription. Medication providers may input their respective offers via the user interface. A selected medication provider may be informed that their offer has been selected via the user interface. The user interface for medication providers may be associated with the user interface for healthcare providers and/or patients. For example, healthcare providers, patients, and medication providers may interact with the prescription processing system 208 via a common website (or two or more related websites) provided by the user interface module 222. A common website may include a healthcare provider portal component, a patient portal component, and a medication provider portal component. The healthcare provider portal component, the patient portal component, and the medication provider portal component may implement the functions described herein associated with the respective parties.

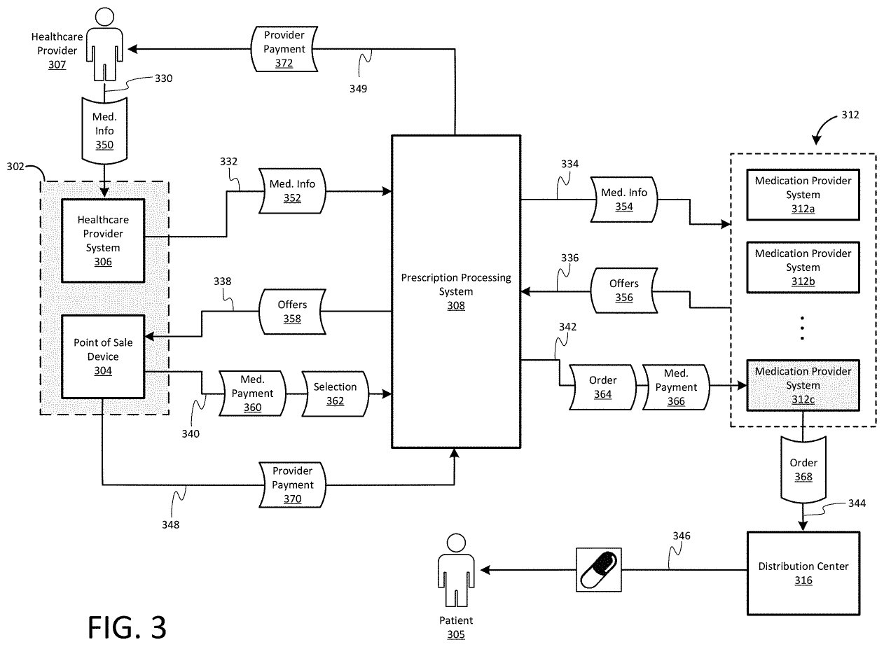

FIG. 3 illustrates a block process diagram relating to the medication selection, ordering, and payment techniques described herein. The system configuration seen in FIG. 3 is similar in some aspects to the system configuration seen in FIG. 1. For example, the premises 302, the healthcare provider system 306, the point of sale device 304, the prescription processing system 308, and the medication provider systems 312 (e.g., 312a-c) may be the same as or similar to, in at least some aspects, the healthcare provider premises 102, the healthcare provider system 106, the point of sale device 104, the prescription processing system 108, and the medication provider systems 112 in FIG. 1, respectively. The prescription processing system 308 may be the same as or similar to, in at least some aspects, the prescription processing system 208 in FIG. 2.

At step 330, a healthcare provider 307 inputs medication information 350 to the healthcare provider system 306 at the premises 302. As an example, the healthcare provider 307 may input the medication information 350 into a personal computer of the healthcare provider system 306. The medication information 350 may be input via a software application running on the healthcare provider system 306 that is associated with the prescription processing system 308. The medication information 350 may be input, via a web browser application running on the healthcare provider system 306, to a website associated with the prescription processing system 308. Although not illustrated as such in FIG. 3 for clarity of illustration, the healthcare provider 307 may be located at the premises 302 at the time of inputting the medication information 350 to the healthcare provider system 306. Likewise, a patient 305 may be located at the premises 302 while the healthcare provider 307 enters the medication information 350.

It will be understood that acts performed by other individuals (e.g., a nurse, medical assistant, or office staff) on the healthcare provider's 307 behalf shall be treated for the purposes of this disclosure as being performed by the healthcare provider 307. Similarly, acts performed by other individuals (e.g., a caregiver, spouse, or relative) on the patient's 305 behalf shall be treated for the purposes of this disclosure as being performed by the patient 305.

The medication information 350 may indicate one or more medications (e.g., prescription medications) that the healthcare provider 307 intends for the patient 305 to take. The medication information 350 may comprise patient information, which may include one or more of personal information (e.g., name, address, date of birth, etc.), medical information, or benefits information. The medical information may comprise medical records, such as electronic medical records. The medical records may indicate current or past medical conditions, height and weight, allergies (e.g., drug allergies), or other medications being taken by the patient 305, for example. The benefits information may indicate a patient's benefits plan (e.g., a drug benefits plan) or health insurance. The benefits information may also indicate any coupons, rebates, or discounts that may be applied to the patient's medication.

In some aspects, the medication information 350 may comprise a prescription for the medication. In other aspects, the medication information 350 may not include a prescription. The medication indicated in the medication information 350 may be an as-yet un-prescribed medication. Additionally or alternatively, the medication information 350 may indicate multiple medications that the healthcare provider contemplates prescribing for the patient 305. For example, the multiple medications may have approximately equal therapeutic value. The patient 305 may wish to receive pricing offers for each medication because their relative costs may fluctuate over time.

In addition to the medication itself, the medication information 350 may indicate several acceptable dosage forms associated with the medication. Dosage form may refer to whether the medication is in the form of a pill, a chewable tablet, a liquid, or a gel capsule, etc. Dosage form may additionally or alternatively refer to the various acceptable combinations of pill (or other unit dose) quantity and amount of active ingredient in each pill. For example, the medication information 350 may indicate a daily dosage as either two 10 mg pills or one 20 mg pill. A quantity of pills (or other unit dose) indicated in the medication information 350 may be additionally or alternatively based on an understanding that the supplied pills will be split by the patient 305. The medication information 350 may indicate several acceptable formulations of a base medication. For example, the medication information 350 may indicate an immediate release formulation of the medication and an extended release formulation of the medication.

At step 332, medication information 352 is sent from the healthcare provider system 306 to the prescription processing system 308. The medication information 352 may be the same as or similar to, in at least some aspects, the medication information 350. The healthcare provider system 306 may reformat or re-package the medication information 352 before it is sent to the prescription processing system 308. For example, the medication information 352 may be in the form of a database or be part of a database sent to the prescription processing system 308. The healthcare provider system 306 may append the patient's 305 medical information stored by the healthcare provider system 306 to the medication information 352. Additionally or alternatively, the healthcare provider system 306 may separately send medication information to the prescription processing system 308. The prescription processing system 308 may receive the medication information 352. For example, the prescription management module of the prescription processing system 308 may receive the medication information 352.

In some aspects, the healthcare provider 307 may identify a particular medication via the medication information 350, which is communicated to the prescription processing system 308 via the medication information 352. The prescription processing system 308 may receive the medication information 352 and determine one or more alternatives with respect to other medications (e.g., therapeutically similar medications), dosage forms, and/or formulations. The one or more alternative medications, dosage forms, and/or formulations may be determined via an artificial intelligence algorithm or logic of the prescription processing system 308. The one or more alternative medications, dosage forms, and/or formulations may be communicated back to the healthcare provider 307 via the healthcare provider system 306.

The healthcare provider system 306 may provide a feature (e.g., a user interface checkbox) for the healthcare provider 307 to opt in or opt out of receiving alternative medications, dosage forms, or formulations. Assuming the healthcare provider 307 has indicated that he or she wishes to receive such alternatives, the healthcare provider 307 may select one or more of the alternatives from those presented. The selected alternatives may comprise a subset of the alternatives presented to the healthcare provider 307. For example, the alternatives may be presented to the healthcare provider 307 via a user interface with a selectable checkbox next to each alternative. The healthcare provider 307 may check off the desired alternatives. The selected alternatives may be communicated back to the prescription processing system 308 (e.g., as a second instance of the medication information 352).

At step 334, medication information 354 is sent to the plurality of medication provider systems 312 associated with a respective plurality of medication providers. The medication information 354 may be the same as or similar to, in at least some aspects, the medication information 350 and/or the medication information 352. The medication information 354 may be sent by the pricing module of the prescription processing system 308. The prescription processing system 308 may determine the medication provider systems 312 that are to receive the medication information 354. For example, the prescription processing system 308 may select medication providers that serve the patient's 305 region or stock the medication indicated in the medication information 352.

The prescription processing system 308 may edit the medication information 352 to generate the medication information 354. For example, the prescription processing system 308 may remove at least some personal information from the medication information 352. Additionally or alternatively, the prescription processing system 308 may add additional information to the medication information 354. For example, the prescription processing system 308 may add benefits information that is stored by the prescription processing system 308. In some aspects, the prescription processing system 308 forwards on the medication information 352 received from the healthcare provider system 306 without substantial change.

The medication information 354 may comprise or be considered as a request for offers for supplying the medication(s) indicated in the medication information 354. The plurality of medication provider systems 312 may each receive the medication information 354.

At step 336, the plurality of medication provider systems 312 send, based on the received medication information 354, respective offers 356 to the prescription processing system 308. An offer 356 may comprise a price for the medication at which the corresponding medication provider offers to supply the medication to the patient 305. The offers 356 may be received by the prescription processing system 308, such as by the pricing module.

If the medication information 354 indicates multiple medications (such as multiple therapeutically similar medications), an offer 356 may comprise a price for each medication. If the medication information 354 indicates multiple acceptable dosage forms, an offer 356 may comprise a price for each dosage form. If the medication information 354 indicates multiple acceptable formulations, an offer may comprise a price for each dosage form. Additionally or alternatively, an offer 356 may comprise several prices for the medication based on any benefits, discounts, rebates, or coupons that may be applied to the medication (or none of the foregoing). For example, an offer 356 may comprise a first price for the medication under the patient's 305 benefits plan, a second price under a manufacturer rebate, and a third price that is the retail price for the medication. Alternatively, prices for multiple medications, multiple dosage forms, or multiple formulations associated with a single medication provider system 312 may be sent as separate offers 356 by the medication provider system 312 rather than as part of the same offer 356.

At step 338, offers 358 from the respective medication providers are sent from the prescription processing system 308 to the point of sale device 304 at the healthcare provider's premises 302. The offers 358 may be the same as or similar to, in at least some aspects, the offers 356. The prescription processing system 308, such as the pricing module, may aggregate the offers 356 from the medication provider systems 312 to determine the offers 358 sent to the point of sale device 304.

The offers 358 may be presented to the patient 305 at the premises 302 via the point of sale device 304. The offers 358 may be presented to the patient 305 via a software application running on the point of sale device 304, such as a specialized software application associated with the prescription processing system 308. Additionally or alternatively, the offers 358 may be presented to the patient 305 via a web browser, such as via a website associated with the prescription processing system 308. “Sending” the offers 358 to the point of sale device 304 may comprise sending a file indicating the offers 358 to the point of sale device 304 and/or causing the offers 358 to be displayed on the point of sale device 304. The patient 305 may use the point of sale device 304 to select an offer from the offers 358, such as via the aforementioned specialized software application or website. In this example, the offer from the medication provider associated with the medication provider system 312c (highlighted in FIG. 3) is selected.

In selecting an offer, the patient 305 may consult with the healthcare provider 307, who may be present with the patient 305 at the point of sale device 304. After the patient 305 has selected the offer, and if not already having done so, the healthcare provider 307 may “write” the prescription for the medication, such as generating an electronic prescription for the medication. In this manner, the patient 305 and healthcare provider 307 may make an informed decision with regard to pricing, medication selection, dosage form, formulation, or various terms associated with a medication provider (e.g., shipping cost and/or shipping time)

At step 340, the selection 362 of the offer is sent from the point of sale device 304 to the prescription processing system 308. The selection 362 may comprise the prescription for the medication. The prescription processing system 308, such as the fulfillment module, may receive and process the selection 362. The selection 362 may indicate not just the medication provider that is to supply the medication (e.g., the medication provider system 312c), but may also indicate a selection of a medication (such as from several therapeutically similar medications), a dosage form, a formulation, or any combination thereof.

Also at step 340, the patient 305 may submit payment for the medication (i.e., the medication payment 360) via the point of sale device 304 at the premises 302. The patient 305 may submit the medication payment 360 by entering a credit card number or bank account information into the point of sale device 304. The patient 305 may submit the medication payment 360 via a credit card reader attached or connected to the point of sale device 304. The medication payment 360 may be sent to the prescription processing system 308 concurrently with the selection 362 or as part of the same data transaction. The prescription processing system 308, such as the payment module, may receive and process the medication payment 360.

At step 342, the prescription processing system 308 may generate an order 364 for the medication (along with any selections regarding dosage form or formulation). The order 364 is sent to the medication provider system 312c associated with the selected offer. The order 364 may comprise the prescription for the medication. The order 364 may additionally comprise personal information, benefits information, and medical information. For example, the order 364 may comprise any patient information required by a pharmacist to fill a prescription.

As described in relation to FIG. 1, the medication provider systems 312 may be integrated, at least in part, with the prescription processing system 308. For example, the medication provider systems 312 may be represented as respective components of the prescription processing system 308 rather than as distinct computing systems. As such, the medication information 354, order 364, and medication payment 366 may be communicated to the medication provider systems 312 internally within the prescription processing system 308, as opposed to external network communications. Likewise, the offers 356 may be communicated from the respective medication provider systems 312 to the prescription processing system 308 as internal communications within the prescription processing system 308. In an aspect, medication providers may access the prescription processing system 308 via a web site or other application interface of the prescription processing system 308. For example, the prescription processing system 308 may comprise a website portal that may be used by medication providers to interact with the prescription processing system 308. For example, medication providers may submit their respective offers 356 via the website portal. The medication providers may receive the medication information 354 and any order 364 and/or medication payment 366 via the website portal.

Also at step 342, payment for the medication, i.e., the medication payment 366, is sent to the medication provider system 312c associated with the selected offer. The medication payment 366 may be based on the medication payment 360 received by the prescription processing system 308 from the point of sale device 304. For example, the prescription processing system 308 may process the patient's 305 credit/debit card information, bank account information, or other source of funding indicated in the medication payment 360. The prescription processing system 308 may pull the patient's 305 payment from the identified funding source. At least a portion of those funds may be transferred to the selected medication provider system 312c as the medication payment 366. In other aspects, the prescription processing system 308 may provide the patient's 305 payment information (e.g., credit card number, etc.) to the selected medication provider system 312c and the medication provider system 312c may pull the appropriate payment from the patient's 305 funding source.

At step 344, the medication provider system 312c generates an order 368 to a distribution center 316 based on the order 364 received from the prescription processing system 308. The distribution center 316 may be associated with the medication provider system 312c. The distribution center 316 may comprise a medication warehouse to store medication and a labeling center to generate labels to be affixed to bottles or other medication packaging.

At step 346, the distribution center 316 provides the medication to the patient 305. For example, the medication may be shipped to the patient's residence. As another example, the medication may be made available for pickup at a brick-and-mortar pharmacy.

Additionally, at step 348, the patient 305 may submit payment, i.e., provider payment 370, to the healthcare provider 307. The provider payment 370 may be for services rendered during the same office visit in which the healthcare provider 307 enters the medication information 350 and the patient 305 selects one of the offers 358. The patient 305 may use the point of sale device's 304 credit card reader or enter credit card or bank account information into the point of sale device 304.

Step 348 may occur at generally the same time as step 340. That is, the patient 305 may enter the selection 362, submit the medication payment 360, and submit the provider payment 370 at the same general time (e.g., within a pre-defined period of time from each other) and via the same device. This may save time and effort for the patient 305 by not having to do each of these steps at separate times and/or via separate devices.

At step 349, the provider payment 372 is transferred to the healthcare provider 307 based on the provider payment 370 submitted to the prescription processing system 308. The provider payment 372 may comprise the payment information submitted by the patient 305 for the healthcare provider 307 to process or the provider payment 372 may be a direct fund transfer to the healthcare provider 307 after the prescription processing system 308 has processed the provider payment 370.

The medication payment 360 for the medication and the provider payment 370 for the healthcare provider's 307 services may be submitted and received at the point of sale device 304 as separate transactions. For example, the patient 305 may have to enter payment information a first time for the medication payment 360 and separately enter payment information a second time for the provider payment 370. Funds at the prescription processing system 308 for the medication payment 366 to the medication provider system 312c and funds at the prescription processing system 308 for the provider payment 372 to the healthcare provider 307 may be processed and maintained separately from one another (e.g., the funds may not be co-mingled).

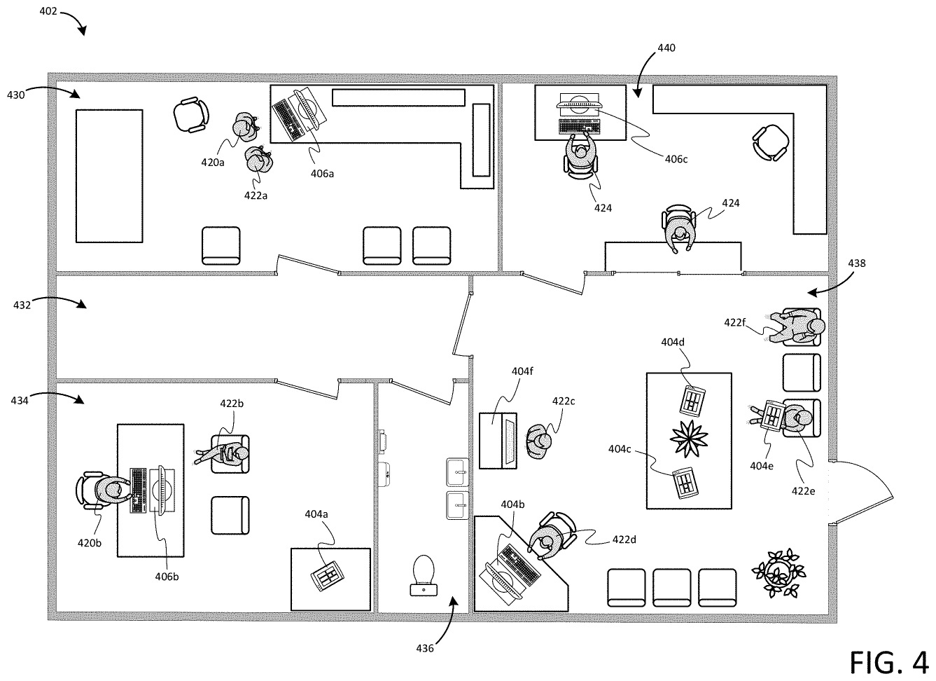

FIG. 4 illustrates a schematic diagram of a healthcare provider premises 402 at which techniques described herein may be implemented, at least in part. For example, a healthcare provider 420a,b (420, in the generic) may determine one or more medications for a patient 422a-f (422, in the generic) and enter the one or more medications into a healthcare provider system 406a-c (406, in the generic). Based on the one or more medications entered into the healthcare provider system 406, and other medication information, one or more offers from medication providers to fill the prescription may be presented to the patient 422 via an associated point of sale device 404a-f (404, in the generic) at the premises 402. The patient 422 may thereby select an offer via the point of sale device 404. The patient 422 may additionally submit a payment for the medication via the point of sale device 404. The patient 422 may additionally submit a payment via the point of sale device 404 for his or her visit to see the healthcare provider 420.

It is initially noted that the premises 402 and the components and people at the premises 402 are not necessarily drawn to scale in FIG. 4. The premises 402 may be the same as or similar to, in at least some aspects, the healthcare provider premises 102, 302 in FIGS. 1 and 3, respectively. The point of sale devices 404 may be the same as or similar to, in at least some aspects, the point of sale device 104, 304 in FIGS. 1 and 3, respectively. The healthcare provider systems 406 may be the same as or similar to, in at least some aspects, the healthcare provider system 106, 306 in FIGS. 1 and 3, respectively.

The premises 402 includes an examination room 430 within which a healthcare provider 420a sees a patient 422a. The examination room 430 includes an examination table, several chairs, and a counter space. A healthcare provider system 406b is placed atop the counter space. The healthcare provider system 406a comprises a personal computer in this instance. The healthcare provider system 406a (e.g., the personal computer) may form just one component of a larger healthcare provider system. For example, the healthcare provider system 406a may be connected to external servers of the larger healthcare provider system. In the scenario shown, the healthcare provider 420a may have entered one or more medications into the healthcare provider system 406a that the healthcare provider 420a contemplates prescribing to the patient 422a. The offers may be returned to the healthcare provider system 406a for review and selection by the patient 422a. The healthcare provider 420a may provide his or her input to the selection since they are both present when the patient 422a receives the offers. In this instance, the healthcare provider system 406a may serve as both a healthcare provider system and a point of sale device. The healthcare provider system 406a may be configured for the patient 422a to submit payment for the medication and/or the visit to the healthcare provider 420a. Although not shown in FIG. 4, a tablet or other handheld computer may be present in the examination room 430 for use by the healthcare provider 420a and/or the patient 422a. This too may act as both a healthcare provider system and a point of sale device.

The premises 402 includes an office 434 of a healthcare provider 420b. The healthcare provider 420b may meet with a patient 422b and provide a consultation to the patient 422b in the office 434. The office 434 includes a desk with a healthcare provider system 406b. The healthcare provider system 406b may comprise a personal computer, which also may form a part of a larger, networked healthcare provider system. The office 434 also includes a table or the like with a point of sale device 404a sitting atop. The point of sale device 404a comprises a tablet or other handheld computing device. The healthcare provider 420b may enter one or more medications into the healthcare provider system 406b for transfer to a prescription processing system (e.g., the prescription processing system 108, 208, 308 of FIGS. 1-3, respectively). One or more offers may be accordingly sent to the point of sale device 404a in the office 434. The patient 422b may consult with the healthcare provider 420b while selecting one of the offers presented on the point of sale device 404a. It will be recalled that the patient 422b may receive two or more offers (even from the same medication provider) that vary with respect to dosage form and/or formulation. Additionally or alternatively, the offers may include offers for several different medications (e.g., therapeutically similar medications). The healthcare provider 420b may further advise on these additional considerations.

The premises 402 includes a reception area/office 440 to receive patients and perform various administrative functions. The reception area/office 440 includes a healthcare provider system 406c, a personal computer in this instance. The reception area/office 440 may be manned by a staff member 424. The staff member 424 may be a medical assistance for the healthcare providers 420a,b. The staff member 424 may be authorized by the healthcare providers 420a,b to enter medication information into the healthcare provider system 406c. This illustrates an example in which the medication information entered by the staff member 424 is treated as if it were entered by one of the healthcare providers 420a,b.

The premises 402 includes a waiting area 438 for patients 422c-f and includes several point of sale devices 404b-f. The waiting area 438 may serve dual purposes. First, incoming patients 422 that have not yet seen a healthcare provider 420 may wait in the waiting area 438 until called for. Second, those patients 422 that have already seen a healthcare provider 420 may use one of the various point of sale devices 404b-f in the waiting area 438 to select an offer for the medication(s) just entered into one of the healthcare provider systems 406a-c during their instant visit to the premises 402 for healthcare. Such a patient 422 may also enter payment, via a point of sale device 404, for the medication according to the selected offer. Additionally or alternatively, a patient 422 may enter payment, via a point of sale device 404, for the healthcare services received during their instant visit to the premises 402. A payment for medication and a payment for healthcare services may be input to and received by a point of sale device 404 as separate transactions.

The point of sales devices 404 in the waiting area 438 include a personal computer 404b, several tablets 404c-e or similar handheld computers, and a kiosk 404f The interior of the kiosk 404f may include a computing device (e.g., similar to a personal computer) and the exterior of the kiosk 404f may be equipped with a display (e.g., a monitor screen) and one or more input devices (e.g., a keyboard and/or a pointing device). The kiosk 404f may be generally configured to restrict access to or protect the internal computing components while still allowing user interaction via the external display and input devices. One or more of the point of sale devices 404 in the waiting area 438 (as well as elsewhere in the premises 402) may be secured to prevent theft or damage to the device. For example, the kiosk 404f may be secured via a cable lock, as well as by its greater weight and bulky dimensions. The personal computer 404b may be similarly secured with a cable lock. The tablets 404c-e may be secured, for example, with a retractable tether. The tablets 404c-e may be additionally or alternatively secured through electronic means. For example, a tablet 404c-e may deactivate or lock when moved a certain distance from the premises 402

The premises 402 additionally includes a hallway 432 and a restroom 436.

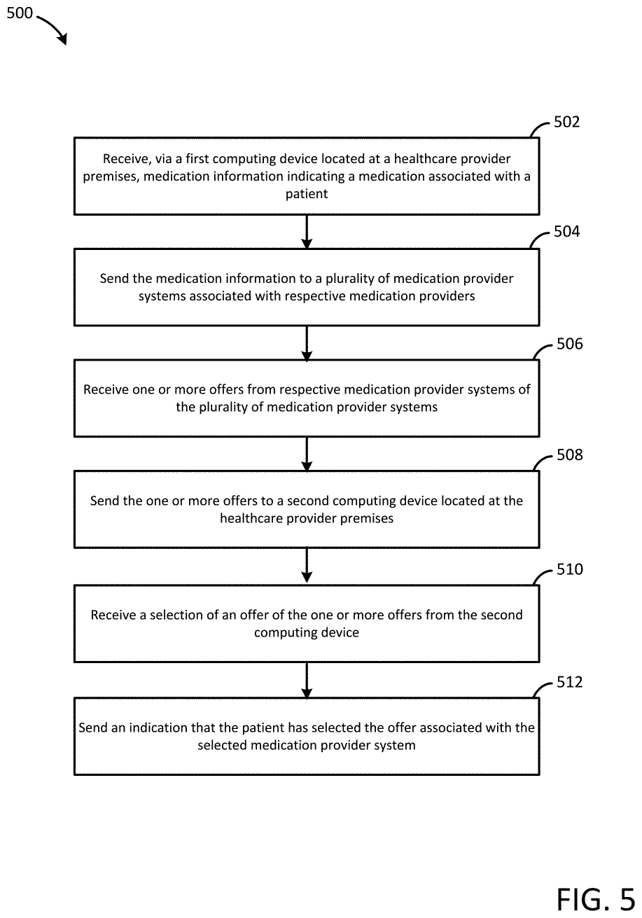

FIG. 5 illustrates an example method 500 relating to selection, ordering, and payment for prescription medication. The method 500 may be performed, at least in part, by a prescription processing system, such as the prescription processing system 108, 208, or 308 of FIGS. 1-3, respectively.

At step 502, medication information is received. The medication information may indicate a medication associated with a patient. The medication information may be received by the prescription processing system. The medication information may be received via a first computing device located at a healthcare provider premises. The first computing device may comprise at least a portion of a healthcare provider system, such as a personal computer or tablet connected to an external network of servers. The medication information may have been input by a healthcare provider into the first computing device. The medication information may have been entered into the first computing device while the patient was at the premises. The medication may be a medication prescribed by the healthcare provider or the medication may be a medication that the healthcare provider considers prescribing to the patient. The medication information may comprise a prescription for the medication. The medication information may be input via a website displayed at the first computing device. Additionally or alternatively, the medication information may be input via a dedicated software application running on the first computing device.

The medication information may indicate two or more medications, such as two or more therapeutically similar medications considered by the healthcare provider for the patient. The medication information may additionally or alternatively indicate two or more acceptable dosage forms of the medication and/or two or more acceptable formulations of the medication. Based on an initial medication input to the first computing device by the healthcare provider, additional or alternative medications, dosage forms, and/or formulations may be suggested by the prescription processing system and presented to the healthcare provider at the first computing device. The additional or alternative medications, dosage forms, and/or formulations indicated in the medication information may be a subset of those suggested by the prescription processing system. For example, the subset may reflect a selection by the healthcare provider from the suggested additional or alternative medications, dosage forms, and/or formulations. The medication information may also include patient information, such as personal information, medical records, or benefits information (e.g., information on the patient's prescription medication benefits plan and/or any coupons, discounts, or rebates available for the patient).

At step 504, the medication information is sent to a plurality of medication provider systems associated with respective medication providers. The medication information may be sent by the prescription processing system to the plurality of medication provider systems. The medication information sent to the plurality of medication provider systems may include, be appended with, or be sent concurrently with additional information associated with the patient, such as personal information, medical records, and/or benefits information. Such additional information may have been stored by the prescription processing system, but not by the healthcare provider system. The prescription processing system may determine the plurality of medication provider systems that are sent the medication information. This may be based on one or more characteristics of the patient and/or one or more characteristics of a medication provider. For example, a medication provider may not serve or ship to the patient's area. As another example, a medication provider may not honor the patient's benefits plan or may be out-of-network under the patient's benefits plan.

At step 506, one or more offers are received from respective medication provider systems of the plurality of medication provider systems. An offer may comprise an offer by the associated medication provider (e.g., associated with a corresponding medication provider system) to fulfill a prescription for the medication. An offer may comprise a medication provider's price for the medication, as well as additional terms of the transaction such as shipping time and cost. The one or more offers may be received by the prescription processing system. A medication provider may provide multiple offers, such as one offer for a first medication and a second offer for a second medication. The first and second medications may have been indicated in the medication information from the healthcare provider system as acceptable, therapeutically similar medications. A first and second offer from a single medication provider may additionally or alternatively relate to one or more of dosage form or formulation. The one or more offers may be received from respective medication provider systems (e.g., medication providers) via a website associated with the prescription processing system. For example, the prescription processing system may provide a website portal for medication providers.

At step 508, the one or more offers are sent to a second computing device located at the healthcare provider premises. The one or more offers may be sent by the prescription processing system. The second computing device may comprise a point of sale device at the healthcare provider premises, such as a personal computer, a tablet or other handheld computer, or a computer kiosk. The second computing device may be secured at the healthcare provider premises. The one or more offers may be received by and presented to the patient via the second computing device (e.g., by the second computing device). The one or more offers may be presented to the patient via a website or a dedicated software application running on the second device. The website used to present the one or more offers may be the same website used by the healthcare provider to enter the medication information. This same website may be additionally used to receive a selection of an offer by the patient via the second computing device.

The healthcare provider may enter the medication information into the healthcare provider system and the patient may be presented with the one or more offers during the same healthcare visit by the patient to the healthcare provider premises. For example, the healthcare provider may enter the medication information while in the examination room or the healthcare provider's office (e.g., with the patient present) and the patient may be presented with (and later select from) the one or more offers via the second computing device (e.g., point of sale device) located in the healthcare provider's waiting area. The healthcare provider entering the medication information and the second computing device presenting the one or more offers may occur within a pre-defined period of time of each other.

At step 510, a selection of an offer of the one or more offers (sent to the second computing device in step 508) is received. The selection of the offer may be received from the second computing device. The selection of the offer may be received by the prescription processing system. The selected offer may be associated with a selected medication provider system of the plurality of medication provider systems. By extension, the selected offer may be associated with a selected medication provider.

Additionally or alternatively, medication payment information may be received by the prescription processing system from the second computing device. The medication payment information may be associated with fulfillment of the prescription for the medication by the selected medication provider. The medication payment information may be used by the prescription processing system or the selected medication provider to effect payment to the selected medication provider for the medication.

Additionally or alternatively, healthcare provider payment info may be received by the prescription processing system from the second computing device. The healthcare provider payment information may be associated with healthcare services received by the patient (e.g., from the healthcare provider) at the healthcare provider premises. The healthcare provider payment information may be used by the prescription processing system and/or healthcare provider to effect payment to the healthcare provider. The healthcare provider payment information and the medication payment information may be input to the second computing device concurrently, such as within a pre-defined period of time of each other or within the same browser session with the website. The medication payment information and the healthcare provider payment may be input to the second computing device as separate transactions.

At step 512, an indication that the patient has selected the offer associated with the selected medication provider system is sent. The indication may be sent to the selected medication provider system. The indication may be sent by the prescription processing system. The indication may comprise an order for the medication. The indication may comprise the prescription for the medication. Medication payment information may be sent to the selected medication provider system appended to, as part of, or concurrently with the indication that the patient has selected the offer. In some aspects, the prescription processing system may process the medication payment information and transfer the resultant payment to the selected medication provider. In some aspects, the indication may not be sent to the selected medication provider system until the medication payment information is received from the patient. In some aspects, the indication may not be sent to the selected medication provider system until payment is cleared for transfer to the selected medication provider.

Having received the indication that the patient has selected the offer associated with the selected medication provider system, the selected medication provider may generate an order to a distribution center. The distribution center may package and label the medication. The distribution center may deliver or otherwise provide the medication to the patient.

One skilled in the art will appreciate that the systems and methods disclosed herein may be implemented via a computing device that may comprise, but are not limited to, one or more processors, a system memory, and a system bus that couples various system components including the processor to the system memory. In the case of multiple processors, the system may utilize parallel computing.

For purposes of illustration, application programs and other executable program components such as the operating system are illustrated herein as discrete blocks, although it is recognized that such programs and components reside at various times in different storage components of the computing device, and are executed by the data processor(s) of the computer. An implementation of service software may be stored on or transmitted across some form of computer readable media. Any of the disclosed methods may be performed by computer readable instructions embodied on computer readable media. Computer readable media may be any available media that may be accessed by a computer. By way of example and not meant to be limiting, computer readable media may comprise “computer storage media” and “communications media.” “Computer storage media” comprise volatile and non-volatile, removable and non-removable media implemented in any methods or technology for storage of information such as computer readable instructions, data structures, program modules, or other data. Exemplary computer storage media comprises, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which may be used to store the desired information and which may be accessed by a computer. Application programs and the like and/or storage media may be implemented, at least in part, at a remote system.

As used in the specification and the appended claims, the singular forms “a,” “an” and “the” include plural referents unless the context clearly dictates otherwise. Ranges may be expressed herein as from “about” one particular value, and/or to “about” another particular value. Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect.

It will be apparent to those skilled in the art that various modifications and variations may be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

1. A method comprising:
receiving, via a first computing device located at premises of a healthcare provider, medication information indicating a medication associated with a patient;
sending the medication information to a plurality of medication provider systems associated with respective medication providers;
receiving one or more offers to fulfill the medication from one or more medication provider systems of the plurality of medication provider systems;
sending the one or more offers to a second computing device located at the premises of the healthcare provider, wherein the one or more offers are presented to the patient via the second computing device;
receiving a selection of an offer of the one or more offers from the second computing device, wherein the offer is associated with a selected medication provider system of the plurality of medication provider systems and the selection of the offer is performed via the second computing device by the patient at the premises of the healthcare provider;
sending, to the selected medication provider system, an indication that the patient has selected the offer associated with the selected medication provider system; and
receiving payment information for the offer associated with the selected medication provider system and for costs associated with a visit to the healthcare provider by the patient, wherein the payment information is received from the patient at the premises of the healthcare provider.

2-3. (canceled)

4. The method of claim 1 further comprising:
transferring payment, based on the payment information, to the medication provider associated with the selected medication provider system.

5. (canceled)

6. The method of claim 1, further comprising:
transferring payment, based on the payment information, to the healthcare provider.

7. The method of claim 1, wherein the second computing device is configured with a card reader device configured to read the payment information.

8. The method of claim 1, wherein the medication information further includes two or more dosage forms of the medication or two or more formulations of the medication.

9. The method of claim 8, wherein receiving the one or more offers comprises receiving a plurality of offers associated with the selected medication provider system that relate to at least one of dosage forms of the medication or formulations of the medication.

10. The method of claim 1, wherein the medication information includes a second medication associated with the patient.

11. The method of claim 10, wherein receiving the one or more offers comprises:
receiving a plurality of offers associated with the selected medication provider system, wherein a first offer of the plurality of offers associated with the selected medication provider system relates to the medication and a second offer of the plurality of offers associated with the selected medication provider system relates to the second medication.

12. The method of claim 1, further comprising:
receiving a prescription for the medication from the first computing device at the premises of the healthcare provider.

13. The method of claim 1, wherein the second computing device is secured at the premises of the healthcare provider.

14. The method of claim 1, wherein the medication information is received via a website, the one or more offers are presented to the patient via the website, and the selection of the offer is received via the website.

15. A system comprising:
a first computing device located at premises of a healthcare provider;
a second computing device located at the premises of the healthcare provider; and
a third computing device located external to the premises of the healthcare provider, wherein the third computing device is configured to:
receive, from the first computing device, medication information indicating a medication associated with a patient,
send the medication information to a plurality of medication provider systems associated with respective medication providers,
receive one or more offers to fulfill the medication from one or more medication provider systems of the plurality of medication provider systems,
send the one or more offers to the second computing device, wherein the one or more offers are presented to the patient via the second computing device,
receive a selection of an offer of the one or more offers from the second computing device, wherein the offer is associated with a selected medication provider system of the plurality of medication provider systems and the selection of the offer is performed by the patient at the premises of the healthcare provider,
send, to the selected medication provider system, an indication that the patient has selected the offer associated with the selected medication provider system, and
receive payment information for the offer associated with the selected medication provider system and for costs associated with a visit to the healthcare provider by the patient, wherein the payment information is received from the patient at the premises of the healthcare provider.

16-17. (canceled)

18. A non-transitory computer readable medium storing instructions that, when executed by one or more processors, cause:
receiving, via a first computing device located at a premises of a healthcare provider, medication information indicating a medication associated with a patient;
sending the medication information to a plurality of medication provider systems associated with respective medication providers;
receiving one or more offers to fulfill the medication from one or more medication provider systems of the plurality of medication provider systems;
sending the one or more offers to a second computing device located at the premises of the healthcare provider, wherein the one or more offers are presented to the patient via the second computing device;
receiving a selection of an offer of the one or more offers from the second computing device, wherein the offer is associated with a selected medication provider system of the plurality of medication provider systems and the selection of the offer is performed via the second computing device by the patient at the premises of the healthcare provider;
sending, to the selected medication provider system, an indication that the patient has selected the offer associated with the selected medication provider system; and
receiving payment information for the offer associated with the selected medication provider system and for costs associated with a visit to the healthcare provider by the patient, wherein the payment information is received from the patient at the premises of the healthcare provider.

19-20. (canceled)

21. The method of claim 1, wherein the medication information is received via the first computing device from the healthcare provider at the premises of the healthcare provider.

22. The method of claim 21, wherein the one or more offers are presented to the patient via the second computing device within a pre-defined period of time since the medication information was received via the first computing device from the healthcare provider.

23. The method of claim 21, wherein the medication information is received via the first computing device from the healthcare provider and the one or more offers are presented to the patient via the second computing device during a same visit by the patient to the premises of the healthcare provider.

24. The method of claim 23, wherein the patient performs the selection of the offer via the second computing device also during the same visit by the patient to the premises of the healthcare provider.

25. The method of claim 1, wherein the premises of the healthcare provider comprises a physician's office.

26. The method of claim 24, wherein the patient provides the payment information via the second computing device also during the same visit by the patient to the premises of the healthcare provider.

27. The method of claim 1, wherein the one or more offers comprise a plurality of offers, each of the plurality of offers originating from a different one of the plurality of medication provider systems.

28. The method of claim 1, wherein the payment information for the offer associated with the selected medication provider system and the payment information for the costs associated with the visit to the healthcare provider by the patient are received as separate transactions.

29. The method of claim 1, wherein the first computing device is a device of the healthcare provider such that the healthcare provider sends the medication information indicating the medication associated with the patient.
